(19) United States
(12) Patent Application Publication
(10) Pub. No.: US 2025/0017861 A1
(43) Pub. Date: Jan. 16, 2025

(54) DIRECT TABLETING AUXILIARY COMPOSITION

(71) Applicant: BASF SE, Ludwigshafen (DE)

(72) Inventors: Silke Gebert, Ludwigshafen (DE); Felicitas Guth, Ludwigshafen (DE); Claudia Scholten, Ludwigshafen (DE); Thorsten Cech, Ludwigshafen (DE); Florian Bang, Ludwigshafen (DE)

(21) Appl. No.: 18/714,342

(22) PCT Filed: Nov. 30, 2022
(86) PCT No.: PCT/EP2022/083789

§ 371 Date: May 29, 2024

(30) Foreign Application Priority Data
Dec. 2, 2021 (EP) 21212043.0

(51) Int. Cl. A61K 9/16

(52) U.S. Cl. CPC A61K 9/1635; A61K 9/1623; A61K 9/1641

(57) ABSTRACT

The present invention relates to a direct tableting auxiliary composition based on lactose powder mixed intimately with a water-soluble polyethylene glycol-polyvinyl alcohol graft copolymeras binder, a lubricants, a colorant and optionally a crosslinked insoluble polyvinylpyrrolidone as tablet disintegrating agent.

The present invention relates to a direct tableting auxiliary composition based on lactose powder mixed intimately with a water-soluble polyethylene glycol-polyvinyl alcohol graft copolymer as binder, a lubricant, a colorant and optionally a crosslinked insoluble polyvinylpyrrolidone as tablet disintegrating agent.

In the pharmaceutical industry the most commonly employed means to deliver APIs (active pharmaceutical ingredients) is the tablet, which may be obtained through the compression of appropriately formulated powders. Conventional, compressible mixtures are typically obtained by the blending of an API and suitable excipient materials. These excipients may include diluents or fillers, binders, or adhesives, disintegrants, glidants or flowability agents, colorants, flavors and mixtures thereof.

Colored tableting aids are very suitable for the manufacture of two-or multilayered tablets. It can be desired to demonstrate the presence of two different types of actives or compositions in one tablet and to increase patient compliance by this visual appearance. A sharp separation of those individual layers is often desired but difficult to achieve. By simple physical mixing of up to 10% by weight of a colorant with a tableting aid, the powder powderous composition does not show a homogeneous appearance but a spotted blend with insufficient opacity (FIG. 1)

In the art, there is however the continuous need for new excipient compositions and for further optimizing the process for producing ready-to-use co-processed excipient compositions. Especially there remains the need in the industry for continuous manufacturing concepts. Continuous manufacturing concepts for pharmaceutical excipients which exhibit a good flowability while simultaneously enabling acceptable ejection forces during tableting and having a high hardness and fast disintegration time of the compressed dosage form. Such a performance is requested for a variety of drug containing tablets e.g. analgesics.

Thus in one aspect the present invention provides a direct tableting auxiliary composition comprising at least one lactose component, at least one water soluble polyethylene glycol-polyvinyl alcohol graft copolymer, at least one lubricant, at least one colorant and optionally at least one water-insoluble crosslinked polyvinylpyrrolidone.

Lactose belongs to the group of disaccharides and consists of the two molecules β-D-galactose and α/β-D-glucose, which are linked together by a β-1,4 glycosidic bond. According to the invention, the lactose component may be an anhydrous lactose or a lactose monohydrate. Lactose monohydrate is preferred, since it is less hygroscopic compared to anhydrous lactose and is thus more suitable in compositions containing water-sensitive pharmaceutically active ingredients. More preferred is a lactose monohydrate having a content of amorphous lactose monohydrate of less than 5% by weight.

Polyethylene glycol-polyvinyl alcohol graft copolymer is a white to yellowish powder that dissolves easily in water with a low viscosity and has the advantage not to be oxygen sensitive and not to build hydrogen peroxides like other polymers do. It is a graft polymer, comprising polyethylene glycol and polyvinyl alcohol, bound in a ratio of 25:75. It has a molecular weight of approximately 45000. The addition of polyethylene glycol-polyvinyl alcohol graft copolymer enables both the particle size distribution to be optimized and unexpectedly, to keep the disintegration time of the tablets low despite the use of a water-soluble binder. The powder of polyethylene glycol-polyvinyl alcohol graft copolymer is marketed as Kollicoat®IR and contains approximately 0.3% colloidal silica. The polyethylene glycol and polyvinyl alcohol graft copolymer used in this invention does not contain any colloidal silica. The direct tableting auxiliary composition does remarkably contain no glidant/flowability agent at all, and still exhibits extraordinarily high flowability.

The crosslinked insoluble polyvinylpyrrolidones are widely used in the pharmaceutical industry because of their swelling properties. They are thus predominantly used as disintegrants in tablets. Furthermore, their application as pharmaceutical excipient is triggered by their ability to hydrophilize insoluble drugs, to stabilize suspensions and to form complexes, as well as their adsorptive properties. According to the invention the crosslinked polyvinylpyrrolidone may be Kollidon CL and/or Kollidon CL-SF and/or Kollidon CL-F, whereby Kollidon CL-F is preferred.

The lubricants used according to the invention are sodium stearyl fumarate, magnesium stearate, stearic acid and/or poloxamer 407 (Kolliphor P 407 micro), preferably it is sodium stearyl fumarate and magnesium stearate and most preferably it is magnesium stearate. The colorants used in this invention are classified in 3 groups A. Organic dyes and their lakes, B. Inorganic or mineral colors and C. Natural colors or vegetable and animal colors.
A.) Organic dyes are synthetic, chemical compounds that exhibit their coloring power or tinctorial strength when dissolved in a solvent, examples are, erythrosine, patent blue V and the azo dyes tartrazine and sunset yellow. Lakes have been defined by the FDA as the “aluminum salts of FD&C water soluble dyes extended on the substratum of alumina”. Lakes, unlike dyes are insoluble and color by dispersion. Consequently, the particle size of lakes is very critical to their coloring capacity and tinctorial strength. Lakes are largely water-insoluble forms of the common synthetic water-soluble dyes. Some examples of aluminum lakes are brilliant blue lake, sunset yellow lake, amaranth lake, allura red lake, indigo carmine lake, quinoline yellow lake.
B.) Possibly the most important application of mineral coloring in a present day medicament is the use of a mixture of red and yellow ferric oxides. Titanium dioxide is used to color and opacify hard gelatine capsules.
C.) Natural colorants or vegetable and animal colorants are a chemically and physically diverse group of materials. Some of these colorants are the products of chemical synthesis rather than extraction from a natural source, for example β-carotene of commerce is regularly synthetic in origin.

From the 3 groups colorants of group A are preferred. Preferred from the group A colorants are FD&C and the D&C dyes, dyes approved for the use in food, drug and cosmetics (FD&C) or drug and cosmetics (D&C).

The direct tableting auxiliary composition according to the invention preferably comprises the lactose component in an amount of 75-98.75% by weight, more preferably 80-95% by weight and even more preferably 83-92% by weight based on the total mass of the composition.

The crosslinked insoluble polyvinylpyrrolidone component is preferably present in an amount of 0-15%-by weight, more preferably 2-12% by weight and even more preferably 3-10% by weight based on the total mass of the composition.

The polyethylene glycol-polyvinyl alcohol graft copolymer may be comprised in the composition in an amount of 0.5-10% by weight, more preferably 1-10% by weight and even more preferably 2-9% by weight based on the total mass of the composition.

The composition of the invention preferably has a total amount of lubricant of 0.5-10% by weight more preferably 1-10% by weight and even more preferably 2-9% by weight based on the total mass of the composition. According to the invention the lubricant is sodium stearyl fumarate, magnesium stearate, stearic acid and/or poloxamer 407 (Kolliphor P 407 micro), preferably it is sodium stearyl fumarate, and magnesium stearate and most preferably it is magnesium stearate.

The colorant is preferably present in an amount of 0.25-12%-by weight, more preferably 1-11%-by weight and even more preferably 2-9%-by weight based on the total mass of the composition, wherein the sum of all components of the composition adds up to 100% by weight

The direct tableting auxiliary composition has a spherical morphology and is preferably present in the form of granules, whereby the mean particle size (D50) of said granules is preferably in the range of from 50-500 μm, more preferably of from 80-300 μm and even more preferably in the range of from 100-250 μm.

The colored composition has a homogeneous and intense color, even when only 0.5%-by weight of colorant are included in the composition (FIG. 2).

During the tableting process, the composition of the invention may be mixed with at least one API with API levels ranging from 1 to 75% by weight.

In another aspect the composition, preferably the granule is free of any API.

In a further aspect of the invention, a method for manufacturing a composition as described above is provided. The method of the invention comprises the steps of (i) providing a solution or suspension comprising at least one polyethylene glycol-polyvinyl alcohol graft copolymer, at least one colorant and at least one lactose in a liquid medium, optionally a crosslinked insoluble polyvinylpyrrolidone, and (ii) spraying the solution or suspension obtained in step (i) in an environment at an increased temperature, optionally at reduced pressure, thereby removing the liquid medium. In step (i), the at least one polyethylene glycol-polyvinyl alcohol graft copolymer component, at least one colorant and the at least one lactose component and optionally at least one crosslinked insoluble polyvinylpyrrolidone are preferably at least partially solved in a liquid medium, such as water, or an organic solvent, such as ethanol, acetic acid and acetone, and mixtures thereof.

In step (i) it is particularly advantageous to incorporate the colorant as a liquid concentrate (dispersed concentrated suspension or concentrated solution).

In step (ii), the solution or suspension obtained in step (i) is sprayed whereby in contrast to the state of the art the lubricant is not applied as solution or suspension but as a solid powder to the surface of the spray dried particles.

It is particularly advantageous that this new process could be integrated into a continuous manufacturing process without additional preparation steps.

In another aspect the present invention relates to a composition as obtained by the above described process. It has turned out that this process results in composition with excellent flowability, a high bulk density and excellent tableting properties and uniform color. In a further aspect the present invention is directed to the use of the composition as described above as an excipient in making oral dosage forms, particularly as a tableting excipient, more particularly as a direct tableting excipient. Due to the excellent flowability and high bulk density, the composition is also very suitable as diluent for binary mixtures with APIs to be filled into hard shell capsules (e.g., hard gelatin capsules).

The invention is further illustrated by the following figures and examples.

### FIGURES

FIG. 1: Visual appearance: physical mixtures of a lactose based tableting auxiliary without colorant and increasing amount of indicotin 85 (E132) from left (0.5% indicotin 85) to right (10% indicotin 85). All samples are white with an increasing amount of colored spots and have an inhomogeneous appearance.

FIG. 2: Visual appearance: comparison of the direct tableting auxiliary composition according to the invention (left) where 0.5% (w/w) indicotin 85 (E132) was included as a colorant (left sample) with a physical mixture of 0.5% indicotin 85 (E132) and an un-colored tableting auxiliary (right sample). The sample according to the invention (left) has an intense and homogeneously distributed color.

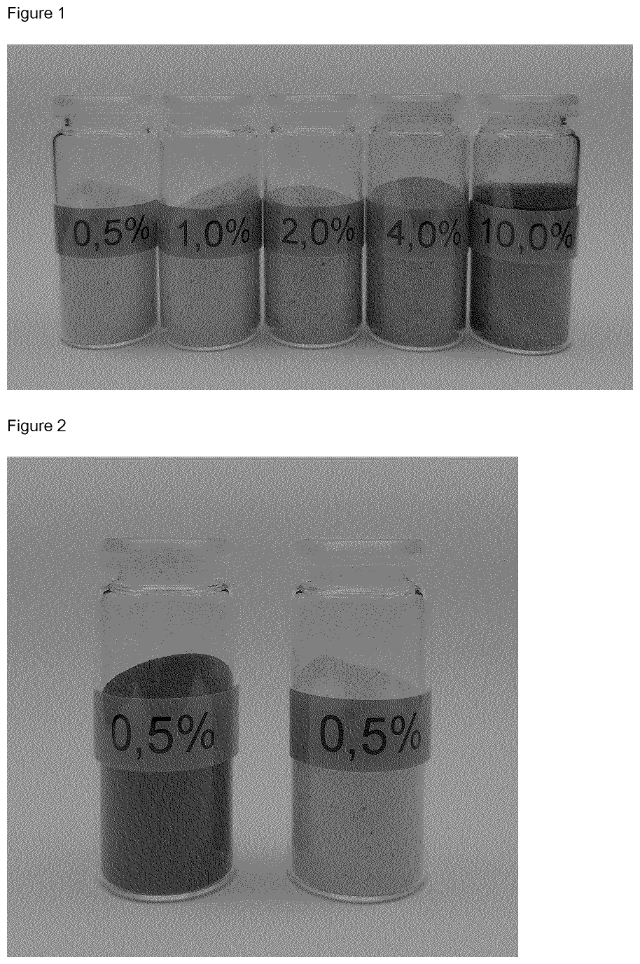

### EXAMPLES

### Methods

The tapped density of the colored direct tableting auxiliary composition was measured in accordance with Chapter 2.9.34 method 2 of the European Pharmacopeia 9.

The bulk density of the colored direct tableting auxiliary composition was measured in accordance with Chapter 2.9.34 method 3 of the European Pharmacopeia 9.

Hausner ratio equals the quotient of tamped density and bulk density.

The packing fraction equals the quotient of bulk density and true density

Flowability and angle of repose of the colored direct tableting auxiliary composition are determined in accordance with Chapter 2.9.16 and Chapter 2.9.36 of the European Pharmacopeia 9.

Particle size distribution (D10, D50, D90) of the direct tableting auxiliary composition were determined using a Malvern Mastersizer 2000.

Tablet hardness was measured in accordance with Chapter 2.9.8 of the European Pharmacopeia 9 using a Sotax HT 100 tablet tester, the tablet hardness being determined successively on 20 tablets with a speed of the test jaw of 120 mm/min.

### Example 1: Manufacturing of the Colored Direct Tableting Auxiliary Composition According to the Invention

A solution of Kollicoat® IR (not containing any colloidal silica) in water, a colorant, and crosslinked insoluble polyvinylpyrrolidone (e.g. Kollidon CL-F) were suspended in water and the suspension cooled down to less than 20° C. It is particularly advantageous to incorporate the colorant as a liquid concentrate (dispersed concentrated suspension or concentrated solution). Under stirring lactose (e.g. GranuLac) was continuously dosed to the suspension. For removing the solvent the obtained suspension was sprayed dried at an inlet air temperature of 155° C.±5° C., outlet air temperature of >80° C., whereby the magnesium stearate was dosed into the spray dryer in dry form and afterwards cooled down, whereby the fines were separated from the granules by a cyclone. The direct tableting auxiliary had the composition shown in Table 1-5.

TABLE 1

Amount in % by weight

| Ingredient | Composition 1 | Composition 2 |
|---|---|---|
| Lactose | 86.25 | 95.25 |
| polyethylene glycol-polyvinyl | 3.5 | 3.5 |
| alcohol graft copolymer | | |
| Puricolor Yellow PYE1-New | 0.25 | 0.25 |
| (suspension) | | |
| crosslinked insoluble | 9 | 0 |
| polyvinylpyrrolidone | | |
| sodium stearyl fumarate | 1 | 1 |

TABLE 2

| | Amount in % by weight | |
|---|---|---|
| | Composition 1 | Composition 2 |
| Ingredient | Lactose D90 53 μm | Lactose D90 92 μm |
| Lactose | 95.0 | 95.0 |
| polyethylene glycol-polyvinyl | 3.5 | 3.5 |
| alcohol graft copolymer | | |
| Luconyl NG Blue 7080 | 0.5 | 0.5 |
| (suspension) | | |
| crosslinked insoluble | 0 | 0 |
| polyvinylpyrrolidone | | |
| sodium stearyl fumarate | 1 | 1 |

TABLE 3

Amount in % by weight

| Ingredient | Composition 1 | Composition 2 |
|---|---|---|
| Lactose | 95.0 | 88.5 |
| polyethylene glycol-polyvinyl | 3.5 | 10.0 |
| alcohol graft copolymer | | |
| Tartrazin 85 (E102) | 0.5 | 0.5 |
| crosslinked insoluble | 0 | 0 |
| polyvinylpyrrolidone | | |
| magnesium stearate | 1 | 1 |

TABLE 4

Amount in % by weight

| Ingredient | Composition 1 | Composition 2 |
|---|---|---|
| Lactose | 95.0 | 95.0-88.5 |
| polyethylene glycol-polyvinyl | 3.5 | 10.0 |
| alcohol graft copolymer | | |
| Indigotin 85 (E132) | 0.5 | 0.5-10 |
| | | *added after |
| | | spray drying |
| crosslinked insoluble | 0 | 0 |
| polyvinylpyrrolidone | | |
| magnesium stearate | 1 | 1 |

TABLE 5
For the same color effect compared to organic dyes a higher
amount of natural colorants is needed. Riboflavin has
a lower opacity than Tartrazin 85 (Gelborange 85 (E110)

Amount in % by weight

| Ingredient | Composition1 | Composition2 |
|---|---|---|
| Lactose | 85.5 | 93.5 |
| polyethylene glycol-polyvinyl | 3.5 | 3.5 |
| alcohol graft copolymer | | |
| Riboflavine | 10.0 | 0 |
| Gelborange 85 (E110) | 0 | 2.0 |
| crosslinked insoluble | 0 | 0 |
| polyvinylpyrrolidone | | |
| magnesium stearate | 1 | 1 |

TABLE 6
The particle size distribution and flowability
of the compositions shown in Table 1-5

| Table | | D50 [μm] | Angle of repose [°] |
|---|---|---|---|
| 1 | Composition 1 | 161 | 26 |
| | Composition 2 | 156 | 27 |
| 2 | Composition 1 | 153 | 27 |
| | Composition 2 | 139 | 28 |
| 3 | Composition 1 | 154 | 27 |
| | Composition 2 | 203 | 26 |
| 4 | Composition 1 | 156 | 27 |
| | Composition 2 | 151 | 27 |
| 5 | Composition 1 | 148 | 27 |
| | Composition 2 | 153 | 27 |

1. A direct tableting auxiliary composition in a form of spherical granules comprising
A) 75-98.75% by weight of a lactose suitable for tableting
B) 0.5-10% by weight of a water-soluble polyethylene glycol-polyvinyl alcohol graft copolymer
C) 0-15% by weight of a crosslinked insoluble polyvinylpyrrolidone
D) 0.25-12% by weight of a colorant
E) 0.5-10% by weight of a lubricant
and wherein a sum of all components A to E adds up to 100% by weight and a mean particle size (D50) of said spherical granules is in a range of from 50 μm to 500 μm.

2. A direct tableting auxiliary composition in a form of spherical granules comprising
A.) 75-98.75% by weight of a lactose suitable for tableting
B.) 0.5-10% by weight of a water-soluble polyethylene glycol-polyvinyl alcohol graft copolymer
C.) 0.25-12% by weight of a colorant
D.) 0.5-10% by weight of a lubricant
and wherein a sum of all components A to D adds up to 100% by weight and a mean particle size (D50) of said spherical granules is in a range of from 50 μm to 500 μm.

3. The direct tableting auxiliary composition according to claim 1 with a mean particle size of 80 μm to 300 μm.

4. The direct tableting auxiliary composition according to claim 1 with a mean particle size of 100 μm to 250 μm.

5. The direct tableting auxiliary composition according to claim 1, wherein the lactose is lactose monohydrate.

6. The direct tableting auxiliary composition according to claim 1, wherein the insoluble polyvinylpyrrolidone is Kollidon CL-SF and/or Kollidon CL-F and/or Kollidon CL.

7. The direct tableting auxiliary composition according to claim 1, wherein the colorant is an organic dye or a lake thereof.

8. The direct tableting auxiliary composition according to claim 7, wherein the colorant is a FD&C or D&C dye.

9. The direct tableting auxiliary composition according to claim 1, wherein the lubricant is sodium stearyl fumarate, magnesium stearate, and/or stearic acid poloxamer 407 (Kolliphor P 407 micro).

10. The direct tableting auxiliary composition according to claim 9, wherein the lubricant is magnesium stearate.

11. The direct tableting auxiliary composition according to claim 1, wherein the lubricant is located on a surface of said granules.

12. The direct tableting auxiliary composition according to claim 1 having an angle of repose of from 25° to 31°.

13. A continuous process for preparing the direct tableting auxiliary composition of claim 1, comprising spray drying the lactose, the polyethylene glycol-polyvinyl alcohol graft copolymer, the colorant suspension, and an optional crosslinked insoluble polyvinylpyrrolidone suspension, whereby the lubricant is dosed in dry powder form.

14. A continuous process for preparing the direct tableting auxiliary composition of claim 2, comprising spray drying the lactose, the polyethylene glycol-polyvinyl alcohol graft copolymer, and the colorant suspension whereby the lubricant is dosed in dry powder form.

15. The process according to claim 13 where the lubricant is added as a dry powder that yields a product where the lubricant adheres to a surface of the spray dried particles.

16. A cosmetic or pharmaceutical preparation, a preparation of agrochemical actives, a preparation in the sector of food, feed and food or feed supplementation comprising a direct tableting auxiliary composition according to claim 1.
